(19) United States
(12) Patent Application Publication
(10) Pub. No.: US 2025/0064966 A1
(43) Pub. Date: Feb. 27, 2025

(54) METHODS OF TREATING SOLID TUMORS AND COMPOSITIONS THEREOF

(71) Applicant: PTM Therapeutics, Inc., San Mateo, CA (US)

(72) Inventor: Tony W. LIANG, San Mateo, CA (US)

(21) Appl. No.: 18/724,530

(22) PCT Filed: Jan. 5, 2023
(86) PCT No.: PCT/US2023/060151

§ 371 Date: Jun. 26, 2024

Related U.S. Application Data
(60) Provisional application No. 63/297,036, filed on Jan. 6, 2022.

(51) Int. Cl. A61K 47/68; A61P 35/00

(52) U.S. Cl. CPC A61K 47/6851; A61K 47/68033; A61P 35/00

(57) ABSTRACT

The present disclosure provides antibody drug conjugates (ADCs) comprising an antibody or antigen binding fragment that binds to sialyl Lewis (sLeA) and sialyl Lewis C (sLeC) coupled to a linker and a cytotoxic or cytostatic agent, as well as methods of treating cancer using such ADCs.

### CROSS-REFERENCE TO RELATED APPLICATIONS

This application claims the benefit of U.S. Provisional Application No. 63/297,036, filed Jan. 6, 2022, which is hereby incorporated by reference in its entirety.

### REFERENCE TO AN ELECTRONIC SEQUENCE LISTING

The contents of the electronic sequence listing (203462000340seqlist.xml; Size: 19,513 bytes; and Date of Creation: Dec. 28, 2022) are herein incorporated by reference in their entirety.

### FIELD

The present disclosure relates to anti-sialyl Lewis A and anti-sialyl Lewis C antibodies and antigen binding fragments thereof that specifically bind to its epitope expressed on cancer cells, and related fusion proteins and antibody-drug conjugates as well as nucleic acids encoding such biomolecules. The present disclosure also relates to uses of such biomolecules for cancer therapy.

### BACKGROUND

Glycans are carbohydrate-based polymers that can be free or attached to proteins (glycoproteins) or lipids (glycolipids). The glycans of glycoproteins are known to be involved in immunity and inflammation, including sialylated glycans on glycoproteins. Studies have shown sialyl Lewis A (sLeA) to be upregulated in concert with CD44v6 in inflamed regions of human colon from patients with ulcerative colitis. Additionally, high expression of sLeA have been described in human pancreatic, colon and gastric cell lines as well as in adenocarcinomas of the colon, stomach and pancreas.

Aberrant glycosylation has also been described as one of the hallmarks of cancer and modulate immune responses. Similar to what has been observed on inflammatory bowel disease tissues, aberrant glycosylation of proteins leads to overexpression of tumor-associated carbohydrate antigens during malignant transformation. Studies have shown that tumor-associated carbohydrate antigens have contributed to various aspects of cancer development and progression, including proliferation, invasion, angiogenesis and metastasis (Fuster, Nat Rev Cancer, 5:526-42 (2005) and Dube, Nat Rev Drug Discovery, 4:477-88 (2005)).

One type of glycosylation are sialic acids and they are typically found to be terminating branches of N-glycans, O-glycans and glycosphingolipids. Within sialic acids, a variety of diversity are created through alpha linkages between the nine-carbon backbone and secondary diversity through modifications at these carbon positions. Examples of sialylated glycosylation found include sLeA, sLeC and sialyl Lewis X.

Studies have shown that altered post-translational modification events, including glycosylation and sialyation, to be associated with many disease states including cancer progression. CA19.9, a carbohydrate antigen, a sialyl Lewis A containing monoganglioside glycolipid, was identified via the CA19.9 mAb and is overexpressed in many gastrointestinal and pancreatic cancers. As such, the detection of CA19.9 antigen in patient serum has been used as a potential adjunct for diagnosis and monitoring of pancreatic and colon cancer, although guidelines from the American Society of Clinical Oncology strongly discourages the use of CA19.9 as a screening test, particularly for pancreatic cancer, because of the high number of false positives.

The present invention addresses the need for an alternative antibody therapy that targets sLeA and which are therapeutically effective in the treatment of solid tumors.

### SUMMARY OF THE INVENTION

The present invention provides antibody drug conjugates (ADCs) comprising an antibody or antigen binding fragment that binds to sialyl Lewis (sLeA) and sialyl Lewis C (sLeC) coupled to a linker and a cytotoxic or cytostatic agent, as well as methods of treating cancer, such as solid tumors, comprising administering an effective amount of such ADCs to a patient in need thereof.

In one aspect, provided herein is an antibody drug conjugate (ADC) comprising: a. an antibody comprising a heavy chain variable region and a light chain variable region, wherein the heavy chain variable region comprises a CDR-H1 comprising the sequence of SEQ ID NO: 12, a CDR-H2 comprising the sequence of SEQ ID NO: 13, and a CDR-H3 comprising the sequence of SEQ ID NO: 14; and wherein the light chain variable region comprises a CDR-L1 comprising a sequence selected from SEQ ID NOs: 15-17, a CDR-L2 comprising the sequence of SEQ ID NO: 18, and a CDR-L3 comprising the sequence of SEQ ID NO: 19; b. a linker; and c. a cytotoxic or cytostatic agent; wherein the cytotoxic or cytostatic agent is linked to the antibody via the linker.

In some embodiments, the heavy chain variable region comprises a CDR-H1 comprising the sequence of SEQ ID NO: 12, a CDR-H2 comprising the sequence of SEQ ID NO: 13, and a CDR-H3 comprising the sequence of SEQ ID NO: 14; and the light chain variable region comprises a CDR-L1 comprising the sequence of SEQ ID NO: 15, a CDR-L2 comprising the sequence of SEQ ID NO:18, and a CDR-L3 comprising the sequence of SEQ ID NO: 19; the heavy chain variable region comprises a CDR-H1 comprising the sequence of SEQ ID NO: 12, a CDR-H2 comprising the sequence of SEQ ID NO: 13, and a CDR-H3 comprising the sequence of SEQ ID NO: 14; and the light chain variable region comprises a CDR-L1 comprising the sequence of SEQ ID NO:16, a CDR-L2 comprising the sequence of SEQ ID NO:18, and a CDR-L3 comprising the sequence of SEQ ID NO: 19; or the heavy chain variable region comprises a CDR-H1 comprising the sequence of SEQ ID NO:12, a CDR-H2 comprising the sequence of SEQ ID NO: 13, and a CDR-H3 comprising the sequence of SEQ ID NO: 14; and the light chain variable region comprises a CDR-L1 comprising the sequence of SEQ ID NO: 17, a CDR-L2 comprising the sequence of SEQ ID NO: 18, and a CDR-L3 comprising the sequence of SEQ ID NO: 19.

In some embodiments, the antibody comprises a heavy chain variable region comprising a sequence selected from SEQ ID NOs: 2-4, and a light chain variable region comprising a sequence selected from SEQ ID NOs: 5-11. In some embodiments, the antibody comprises: a heavy chain variable region comprising the sequence of SEQ ID NO: 2, and a light chain variable region comprising the sequence of SEQ ID NO: 5; a heavy chain variable region comprising the sequence of SEQ ID NO: 2, and a light chain variable region comprising the sequence of SEQ ID NO: 6; a heavy chain variable region comprising the sequence of SEQ ID NO: 2, and a light chain variable region comprising the sequence of SEQ ID NO: 7; a heavy chain variable region comprising the sequence of SEQ ID NO: 2, and a light chain variable region comprising the sequence of SEQ ID NO: 8; a heavy chain variable region comprising the sequence of SEQ ID NO: 3, and a light chain variable region comprising the sequence of SEQ ID NO: 5; a heavy chain variable region comprising the sequence of SEQ ID NO: 3, and a light chain variable region comprising the sequence of SEQ ID NO: 6; a heavy chain variable region comprising the sequence of SEQ ID NO: 3, and a light chain variable region comprising the sequence of SEQ ID NO: 7; or a heavy chain variable region comprising the sequence of SEQ ID NO: 3, and a light chain variable region comprising the sequence of SEQ ID NO: 8.

In some embodiments, the antibody binds to sialyl Lewis A (sLeA) and sialyl Lewis C (sLeC). In some embodiments, the antibody does not bind to sialyl Lewis X (sLeX). In some embodiments, the binding affinity of the antibody to sLeA is of KD of about 750 μM or less, and the binding affinity of the antibody to sLeC is of KD of about 550 μM or less. In some embodiments, the binding affinity of the antibody to sLeA is of KD of about 100 μM or less, and the binding affinity of the antibody to sLeC is of KD of about 110 μM or less.

In some embodiments, the antibody is a humanized antibody. In some embodiments, the antibody is of the IgA, IgD, IgE, IgG, or IgM class. In some embodiments, the antibody is of the IgG class and has an IgG1, IgG2, IgG3, or IgG4 isotype. In some embodiments, the antibody is of the IgG class and has a human IgG1 isotype. In some embodiments, the antibody is an antibody fragment comprising an antigen binding portion. In some embodiments, the antibody fragment is a scFv, (scFv) 2, Fab, Fab′, or F(ab′)2 fragment.

In some embodiments, the linker is a cleavable linker or a non-cleavage linker. In some embodiments, the linker is a valine-citrulline (v-c) linker, a beta-glucuronide linker, or a succinimidyl-4-(N-maleimidomethyl cyclohexane)-1-carboxylate (SMCC) linker.

In some embodiments, the cytotoxic or cytostatic agent is a microtubule-disrupting agent or a DNA-damaging agent. In some embodiments, the cytotoxic or cytostatic agent is a radionuclide, an alkylating agent, a topoisomerase I inhibitor, a topoisomerase II inhibitor, a DNA intercalating agent, a RNA/DNA antimetabolite, a cell cycle modulator, a kinase inhibitor, a protein synthesis inhibitor, a histone deacetylase inhibitor, a mitochondria inhibitor, or an antimitotic agent. In some embodiments, the cytotoxic or cytostatic agent is a microtubule-disrupting agent selected from an auristatin or a maytansinoid. In some embodiments, the auristatin is an analogue of dolastin 10. In some embodiments, the analogue of dolastin 10 is monomethyl auristatin E (MMAE) or monomethyl auristatin F (MMAF). In some embodiments, the maytansinoid is mertansine (DM1) or ravtasine (DM4). In some embodiments, the cytotoxic or cytostatic agent is a DNA-damaging agent selected from a calicheamicin, duocarmycin, or doxorubicin. In some embodiments, the linker is an SMCC linker and the cytotoxic or cytostatic agent is DM1.

In some embodiments, the ADC further comprises a spacer between the linker and the antibody, and/or a spacer between the linker and the cytotoxic or cytostatic agent.

In another aspect, provided herein is a method of treating cancer, comprising administering to a patient in need thereof an effective amount of any of the antibody drug conjugates (ADCs) provided herein. In some embodiments, the cancer is a solid tumor. In some embodiments, the cancer is a colon carcinoma, a colon adenocarcinoma, a small intestine cancer, a small intestine adenocarcinoma, a skin squamous carcinoma, a rectal cancer, a thyroid cancer, a lung cancer, an esophagus cancer, a colon cancer, an ovarian cancer, a prostate cancer, a liver cancer, a pancreatic cancer, a uterine cancer, a bladder cancer, a stomach cancer, a cervical cancer, a gallbladder cancer, an endometrial cancer, or a breast cancer. In some embodiments, the breast cancer is an invasive ductal or invasive lobular carcinoma. In some embodiments, the esophagus cancer is an adenocarcinoma or a squamous cell carcinoma. In some embodiments, the liver cancer is a cholangiocellular carcinoma. In some embodiments, the ovarian cancer is a mucinous adenocarcinoma. In some embodiments, the rectal cancer is an adenocarcinoma or a rectal cancer lymph node metastasis. In some embodiments, the stomach cancer is an adenocarcinoma. In some embodiments, the cervical cancer is a cervix squamous cell carcinoma. In some embodiments, the endometrial cancer is an endometrioid adenocarcinoma. In some embodiments, the small intestine cancer is a small intestine cancer lymph node metastasis. In some embodiments, the gallbladder cancer is an adenocarcinoma. In some embodiments, the patient is a human.

In another aspect, provided herein is a pharmaceutical composition comprising any of the ADCs disclosed herein and a pharmaceutically acceptable carrier. In another aspect, provided herein is a kit comprising any of the ADCs disclosed herein and an optional pharmaceutically acceptable carrier. In some embodiments, the kit further comprises a package insert comprising instructions for administration of the ADC to treat a cancer in a patient in need thereof. In some embodiments, the cancer is a solid tumor. In some embodiments, the cancer is a colon carcinoma, a colon adenocarcinoma, a small intestine cancer, a small intestine adenocarcinoma, a skin squamous carcinoma, a rectal cancer, a thyroid cancer, a lung cancer, an esophagus cancer, a colon cancer, an ovarian cancer, a prostate cancer, a liver cancer, a pancreatic cancer, a uterine cancer, a bladder cancer, a stomach cancer, a cervical cancer, a gallbladder cancer, an endometrial cancer, or a breast cancer. In some embodiments, the breast cancer is an invasive ductal or invasive lobular carcinoma. In some embodiments, the esophagus cancer is an adenocarcinoma or a squamous cell carcinoma. In some embodiments, the liver cancer is a cholangiocellular carcinoma. In some embodiments, the ovarian cancer is a mucinous adenocarcinoma. In some embodiments, the rectal cancer is an adenocarcinoma or a rectal cancer lymph node metastasis. In some embodiments, the stomach cancer is an adenocarcinoma. In some embodiments, the cervical cancer is a cervix squamous cell carcinoma. In some embodiments, the endometrial cancer is an endometrioid adenocarcinoma. In some embodiments, the small intestine cancer is a small intestine cancer lymph node metastasis. In some embodiments, the gallbladder cancer is an adenocarcinoma. In some embodiments, the patient is a human.

In another aspect, provided herein is a method of making an antibody drug conjugate (ADC), the method comprising conjugating an antibody to a cytotoxic or cytostatic agent via a linker, wherein the antibody comprises a heavy chain variable region and a light chain variable region, wherein the heavy chain variable region comprises a CDR-H1 comprising the sequence of SEQ ID NO: 12, a CDR-H2 comprising the sequence of SEQ ID NO:13, and a CDR-H3 comprising the sequence of SEQ ID NO:14; and wherein the light chain variable region comprises a CDR-L1 comprising a sequence selected from the group consisting of SEQ ID NOs: 15-17, a CDR-L2 comprising the sequence of SEQ ID NO: 18, and a CDR-L3 comprising the sequence of SEQ ID NO: 19. In some embodiments, the heavy chain variable region comprises a CDR-H1 comprising the sequence of SEQ ID NO: 12, a CDR-H2 comprising the sequence of SEQ ID NO:13, and a CDR-H3 comprising the sequence of SEQ ID NO: 14, and the light chain variable region comprises a CDR-L1 comprising the sequence of SEQ ID NO: 15, a CDR-L2 comprising the sequence of SEQ ID NO: 18, and a CDR-L3 comprising the sequence of SEQ ID NO: 19; the heavy chain variable region comprises a CDR-H1 comprising the sequence of SEQ ID NO: 12, a CDR-H2 comprising the sequence of SEQ ID NO:13, and a CDR-H3 comprising the sequence of SEQ ID NO: 14, and the light chain variable region comprises a CDR-L1 comprising the sequence of SEQ ID NO:16, a CDR-L2 comprising the sequence of SEQ ID NO: 18, and a CDR-L3 comprising the sequence of SEQ ID NO: 19; or the heavy chain variable region comprises a CDR-H1 comprising the sequence of SEQ ID NO: 12, a CDR-H2 comprising the sequence of SEQ ID NO: 13, and a CDR-H3 comprising the sequence of SEQ ID NO:14, and the light chain variable region comprises a CDR-L1 comprising the sequence of SEQ ID NO: 17, a CDR-L2 comprising the sequence of SEQ ID NO: 18, and a CDR-L3 comprising the sequence of SEQ ID NO: 19. In some embodiments, the antibody comprises a heavy chain variable region comprising a sequence selected from the group consisting of SEQ ID NOs: 2-4, and a light chain variable region comprising a sequence selected from the group consisting of SEQ ID NOs: 5-11. In some embodiments, the antibody binds to sialyl Lewis A (sLeA) and sialyl Lewis C (sLeC). In some embodiments, the antibody does not bind to sialyl Lewis X (sLeX). In some embodiments, the binding affinity of the antibody to sLeA is of Kp of about 100 μM or less, and the binding affinity of the antibody to sLeC is of Kp of about 110 μM or less. In some embodiments, the antibody is a humanized antibody. In some embodiments, the antibody is of the IgA, IgD, IgE, IgG, or IgM class. In some embodiments, the antibody is of the IgG class and has an IgG1, IgG2, IgG3, or IgG4 isotype. In some embodiments, the antibody is of the IgG class and has a human IgG1 isotype. In some embodiments, the antibody is an antibody fragment comprising an antigen binding portion. In some embodiments, the antibody fragment is a scFv, (scFv) 2, Fab, Fab′, or F(ab′)2 fragment. In some embodiments, the linker is a cleavable linker or a non-cleavage linker. In some embodiments, the linker is a valine-citrulline (v-c) linker, a beta-glucuronide linker, or a succinimidyl-4-(N-maleimidomethyl cyclohexane)-1-carboxylate (SMCC) linker. In some embodiments, the cytotoxic or cytostatic agent is a microtubule-disrupting agent or a DNA-damaging agent. In some embodiments, the cytotoxic or cytostatic agent is a radionuclide, an alkylating agent, a topoisomerase I inhibitor, a topoisomerase II inhibitor, a DNA intercalating agent, a RNA/DNA antimetabolite, a cell cycle modulator, a kinase inhibitor, a protein synthesis inhibitor, a histone deacetylase inhibitor, a mitochondria inhibitor, or an antimitotic agent. In some embodiments, the cytotoxic or cytostatic agent is a microtubule-disrupting agent selected from the group consisting of an auristatin and a maytansinoid. In some embodiments, the auristatin is an analogue of dolastin 10. In some embodiments, the analogue of dolastin 10 is monomethyl auristatin E (MMAE) or monomethyl auristatin F (MMAF). In some embodiments, the maytansinoid is mertansine (DM1) or ravtasine (DM4). In some embodiments, the cytotoxic or cytostatic agent is a DNA-damaging agent selected from the group consisting of a calicheamicin, duocarmycin, and doxorubicin. In some embodiments, the linker is an SMCC linker and the cytotoxic or cytostatic agent is DM1. In some embodiments, the ADC further comprises a spacer between the linker and the antibody, and/or a spacer between the linker and the cytotoxic or cytostatic agent. In another aspect, provided herein is an ADC produced by any of the methods of making an ADC described herein.

### BRIEF DESCRIPTION OF THE DRAWINGS

FIG. 1A illustrates in vitro binding of antibody clone 4772 and hIgG antibodies on T84 and U87-MG cells. FIG. 1B shows the effects of an ADC complex (clone 4772-SMCC-DM1, or 4772-DM1), hIgG-SMCC-DM1 (hIgG-DM1), antibody clone 4772 sham conjugation (4772-DMSO) or antibody clone 4772 alone at a range of 0 to 20 μg/ml of antibody on U87-MG cells at 5000 cells/well after 72 hours of treatment. FIGS. 1C, 1D, and 1E show the effects of an ADC complex (clone 4772-SMCC-DM1, or 4772-DM1), hIgG-SMCC-DM1 (hIgG-DM1), antibody clone 4772 sham conjugation (4772-DMSO) or antibody clone 4772 alone at a range of 0 to 20 μg/ml of antibody on T84 cells at 5000 cells/well, 10,000 cells/well and 20,000 cells/well, respectively, after 72 hours of treatment.

FIG. 2A illustrates binding of antibody clone 4772 and hIgG control antibody on normal colon tissue and colon adenocarcinoma tissue at 10 μg/ml. FIG. 2B illustrates binding of antibody clone 4772 on normal small intestine tissue and small intestine adenocarcinoma tissue.

FIG. 3 illustrates binding of antibody clone 4772 and hIgG control on colon adenocarcinoma tissue, tumor match lymph node metastasis and tumor adjacent tissue.

### DETAILED DESCRIPTION OF THE INVENTION

As used herein, the articles “a” and “an” refer to one or more than one (i.e., at least one) of the grammatical object of the article. By way of example, “an element” means one element or more than one element.

The term “antibody” is intended to encompass antibodies, fragments, specified portions and variants thereof, including single chain antibodies and fragments thereof, derived from an antibody of the present invention. Antibodies include antibody fragments, antibody variants, monoclonal antibodies, polyclonal antibodies, and recombinant antibodies. Antibodies can be generated in mice, rats, rabbits, or humans.

The antibodies can be full-length or can comprise a fragment (or fragments) of the antibody having an antigen portion, including, but not limited to, Fab, Fab′, and F(ab′)₂, facb, pFc¹, Fd, dAb fragment, an isolated CDR, diabodies, triabodies, tetrabodies, linear antibodies, single-chain antibody molecules, bispecific and multi-specific antibodies formed from antibody fragments.

In some embodiments, the antibodies comprise all or a portion of a constant region of an antibody. The constant region is an isotype selected from IgA (e.g., IgA1 or IgA2), IgD, IgE, IgG (e.g., IgG1, IgG2, IgG3, or IgG4), IgM. As used herein, the “constant region” of an antibody includes the natural constant region, allotypes or natural variants, such as D356E and L358M or A431G in human IgG1. See, e.g., Jefferies and Lefranc, MAbs, 1 (4): 332-338 (July-August 2009).

The term “monoclonal antibody” as used herein is not limited to antibodies produced through hybridoma technology. A monoclonal antibody is derived from a single clone, including any eukaryotic, prokaryotic, or phage clone, by any means available or known in the art. Monoclonal antibodies of the present invention can be prepared using a wide variety of techniques known in the art including the use of hybridoma, recombinant and phage display technologies, or a combination thereof.

The term “chimeric” antibody as used herein refers to an antibody having variable sequences derived from a non-human immunoglobulin, such as a rat or a mouse antibody, and a human immunoglobulin constant region, typically chosen from a human immunoglobulin template. Methods for producing chimeric antibodies are known in the art.

“Humanized” form of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody can also comprise at least a portion of an immunoglobulin constant region (Fc), typically that or a human immunoglobulin consensus sequence. Methods of antibody humanization are known in the art.

An “effective amount” as used herein, refers to a dose of the antibody or pharmaceutical composition that is sufficient to reduce the symptoms and signs of diseases of the digestive system, including inflammatory bowel disease or cancers of the digestive system. Such symptoms of inflammatory bowel disease include diarrhea, weight loss, bloody diarrhea, bloody stool, pain, anemia, fatigue, rectal bleeding, and abdominal cramps. Symptoms of cancers of the digestive system include weight loss, pain and detectable mass, either clinically as a palpable mass or radiologically or through other imaging techniques. The term “effective amount” and “therapeutically effective amount” are used interchangeably. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic or other effective agents, and a single agent may be considered to be given in an effective amount if, in conjunction with one or more other agents, a desirable result may be or is achieved. While individual needs vary, determination of optimal ranges of effective amounts of each component is within the skill of the art. Typical dosages comprise 0.1 to 100 mg/kg/body weight. The preferred dosages comprise 1 to 100 mg/kg/body weight. The most preferred dosages comprise 10 to 100 mg/kg/body weight.

The terms “subject”, “individual”, or “patient” can refer to a vertebrate having a disease of the digestive system, such as a vertebrate having inflammatory bowel disease or cancer of the digestive system, or a vertebrate having a cancer, such as any cancer known in the art or described herein. Subjects, individuals, or patients include all warm-blooded animals, such as mammals, such as a rodent, preferably a primate or non-human primate, and more preferably, a human. The terms subject, individual or patient include domesticated animals, such as cats, dogs, etc., livestock (for example, cattle, horses, pigs, sheep, goats, etc.), and laboratory animals (for example, mouse, rabbit, rat, gerbil, guinea pig, etc.). Thus, veterinary uses and medical or pharmaceutical formulations are contemplated herein.

One of skill in the art will recognize that antibodies are “modular” in nature. Throughout the disclosure, various specific embodiments of the various “modules” composing the antibodies of the present invention are described. As specific non-limiting examples, various embodiments of variable heavy chain CDRs, variable heavy chains, variable light chain CDRs, and variable light chains are described. It is intended that all of the specific embodiments may be combined with each other as though each specific combination were explicitly described individually.

Humanized antibodies of the present invention can comprise a heavy chain variable region from Table 1 below and additionally, can comprise a light chain variable region from Table 2 below. Such variable regions can be incorporated into a human IgG1 backbone using methods that are well known in the art.

TABLE 1
Heavy Chain Variable Region Sequences

| HvCh1 | SEQ | EVQLVESGGGLVQPGGSLRLSCAASGFTFSTNAMS |
|---|---|---|
| | ID | WVRQAPGKGLEWVSRLRPKSDNYATYYADSVKG |
| | NO: | RFTISRDNSKNTLYLQMNSLRAEDTAVYYCAKGT |
| | 1 | GFWGQGTTVTVSS |
| HvCh2 | SEQ | EVQLVESGGGLVQPGGSLRLSCAASGFTFSTNAMS |
| | ID | WVRQAPGKGLEWVARLRPKSDNYATYYADSVKG |
| | NO: | RFTISRDNSKNTLYLQMNSLRAEDTAVYYCVTGT |
| | 2 | GFWGQGTTLTVSS |
| HvCh3 | SEQ | EVQLVESGGGLVQPGGSLRLSCAASGFTFSTNAMSWVR |
| | ID | QAPGKGLEWVARLRPKSDNYATYYADSVKGRFTISRD |
| | NO: | DSKNTLYLQMNSLRAEDTAVYYCVTGTGFWGQGTTLT |
| | 3 | VSS |
| HvCh4 | SEQ | EVQLVESGGGLVQPGGSLRLSCAASGFTFSTNAMS |
| | ID | WVRQAPGKGLEWVARLRPKSDNYATYYADSVKG |
| | NO: | RFTISRDDSTSTLYLQMNSLRAEDTAVYYCVTGTG |
| | 4 | FWGQGTTLTVSS |

TABLE 2
Light Chain Variable Region Sequences

| | | |
|---|---|---|
| LiCh1 | SEQ ID | DIVMTQSPDSLAVSLGERATINCKSSQSLLNSGNQ |
| | NO: 5 | KNYLTWYQQKPGQPPKLLIYWTSTRESGVPDRFSG |
| | | SGSGTDFTLTISSLQAEDVAVYYCONDYTSPYTFG |
| | | QGTKLEIK |
| LiCh2 | SEQ ID | DIVMTQSPDSLAVSLGERVTMNCKSSQSLLNSGNQ |
| | NO: 6 | KNYLTWYQQKPGQPPKLLIYWTSTRESGVPDRFSG |
| | | SGSGTDFTLTISSVQAEDVAVYYCONDYTSPYTFG |
| | | QGTKLEIK |
| LiCh3 | SEQ ID | DIVMTQSPDSLAVSLGERATINCKSSQSLLNSGNQ |
| | NO: 7 | KNYLTWYQQKPGQPPKLLFYWTSTRESGVPDRFSG |
| | | SGSGTDFTLTISSLQAEDVAVYYCONDYTSPYTFG |
| | | QGTKLEIK |
| LiCh4 | SEQ ID | DIVMTQSPDSLAVSLGERVTMNCKSSQSLLNSGNQ |
| | NO: 8 | KNYLTWYQQKPGQPPKLLFYWTSTRESGVPDRFSG |
| | | SGSGTDFTLTISSVQAEDVAVYYCONDYTSPYTFG |
| | | QGTKLEIK |
| LiCh5 | SEQ ID | DIVMTQSPDSLAVSLGERVTMNCKSSQSLLNSGNQ |
| | NO: 9 | KNYLTWYQQKPGQPPKLLFYWTSTRESGVPDRFSG |
| | | SGSGTDFTLTISSVQAEDLAVYYCONDYTSPYTFG |
| | | QGTKLEIK |
| LiCh6 | SEQ ID | DIVMTQSPDSLAVSLGERVTMNCKSSQSLLQSGNQ |
| | NO: 10 | KNYLTWYQQKPGQPPKLLFYWTSTRESGVPDRFSG |
| | | SGSGTDFTLTISSVQAEDLAVYYCONDYTSPYTFG |
| | | QGTKLEIK |
| LiCh7 | SEQ ID | DIVMTQSPDSLAVSLGERVTMNCKSSQSLLSSGNQ |
| | NO: 11 | KNYLTWYQQKPGQPPKLLFYWTSTRESGVPDRFSG |
| | | SGSGTDFTLTISSVQAEDLAVYYCONDYTSPYTFG |
| | | QGTKLEIK |

In other embodiments, the humanized antibodies of the present invention can comprise heavy and light chain CDR sequences selected from Tables 3 and 4 below:

TABLE 3
Heavy Chain CDR sequences

| | | |
|---|---|---|
| H-CDR1 | SEQ ID NO: 12 | GFTFSTNAMS |
| H-CDR2 | SEQ ID NO: 13 | RLRPKSDNYATY |
| H-CDR3 | SEQ ID NO: 14 | VTGTGF |
| H-CDR3B | SEQ ID NO: 20 | AKGTGF |

TABLE 4
Light Chain CDR sequences

| | | |
|---|---|---|
| L-CDR1 | SEQ ID NO: 15 | KSSQSLLNSGNQKNYLT |
| L-CDR1B | SEQ ID NO: 16 | KSSQSLLQSGNQKNYLT |
| L-CDR1C | SEQ ID NO: 17 | KSSQSLLSSGNQKNYLT |
| L-CDR2 | SEQ ID NO: 18 | WTSTRES |
| L-CDR3 | SEQ ID NO: 19 | QNDYTSPYT |

The present invention includes antibodies and fragments that specifically binds to sLeA and sLeC, but not sialyl Lewis X (sLeX), compositions comprising antibodies, polynucleotides encoding anti-sLeA/sLeC, but not sLeX antibodies, polynucleotides encoding such antibodies, methods and compositions useful for making such antibodies and binding fragments, and various methods using the same. Glycan structures of sLeA, sLeC, and sLeX are shown in Table 5 below.

TABLE 5
Glycan structure of sialyl Lewis A,
sialyl Lewis C, and sialyl Lewis X

| Glycan name | Glycan structure |
|---|---|
| sialyl Lewis A (sLeA) | NeuAc α2,3 Gal β1,3 [Fucα1,4] GlcNAc |
| sialyl Lewis C (sLeC) | NeuAc α2,3 Gal β1,3 GlcNAc |
| sialyl Lewis X (sLeX) | NeuAc α2,3 Galβ1,4 [Fucα1,3] GlcNAc |

Antibodies against carbohydrate/glycans can be unsuitable for pharmaceutical development because of its generally lower binding affinity. Methods for determining binding affinities for antibodies are known in the art. Generally, the binding affinity to a particular target or substrate is determined by the relationship between the on rate (k_{α}(M⁻¹s⁻¹)) and off rate (k_{d}(s⁻¹)) to produce an equilibrium dissociation constant (K_{D}). The lower the K_{D}, the higher the affinity. Examples of methods for determining antibody affinity include assays utilizing the Octet system (Fortébio) or the Biacore system (GE Healthcare) or other systems instrumentation that determine association and dissociation constants. Accordingly, the present invention comprises antibodies having binding affinity to sLeA of a K_{D} of about 750 μM or less, about 725 μM or less, about 700 μM or less, about 675 μM or less, about 650 μM or less, about 625 μM or less, about 600 μM or less, about 575 μM or less, about 550 μM or less, about 525 μM or less, or about 500 μM or less; and binding affinity to sLeC of a K_{D} of about 550 μM or less, about 525 μM or less, about 500 μM or less, about 475 μM or less, about 450 μM or less, about 425 μM or less, about 400 μM or less, about 375 μM or less, about 350 μM or less, about 325 μM or less, or about 300 μM or less; and no binding to sLeX. In non-limiting examples, antibodies of the present invention comprise a binding affinity to sLeA of a K_{D} of 100 μM or less, 90 μM or less, 80 μM or less, 70 μM or less, 60 μM or less, 50 μM or less, 40 μM or less, 30 μM or less, 20 μM or less, 10 μM or less, 1 μM or less, or 0.1 μM or less; and a binding affinity to sLeC of a K_{D} of 110 μM or less, 100 μM or less, 90 μM or less, 80 μM or less, 70 μM or less, 60 μM or less, 50 μM or less, 40 μM or less, 30 μM or less, 20 μM or less, 10 μM or less, 1 μM or less, or 0.1 μM or less; and no binding to sLeX. In one embodiment, antibodies that are useful in the present invention have a binding affinity to their glycan targets of a K_{D} of about 731.7 μM or less for sLeA and a K_{D} of about 538 μM or less for sLeC and no binding to sialyl Lewis X (sLeX). In one embodiment, antibodies that are useful in the present invention have a binding affinity to their glycan targets of a K_{D} of about 81.3 μM or less for sLeA and a K_{D} of about 103.7 μM or less for sLeC and no binding to sialyl Lewis X (sLeX). In one embodiment, antibodies that are useful in the present invention have a binding affinity to their glycan targets of a K_{D} of about 48.3 μM or less for sLeA and a K_{D} of about 77.1 μM or less for sLeC and no binding to sialyl Lewis X (sLeX). In one embodiment, antibodies that are useful in the present invention have a binding affinity to their glycan targets of a K_{D} of about 41 μM or less for sLeA and a K_{D} of about 70 μM or less for sLeC and no binding to sialyl Lewis X (sLeX). In some embodiments, an antibody of the present disclosure does not bind to a particular target (e.g., sLeX) if the binding affinity of the antibody to the target is of a K_{D} of about 1 mM or higher.

Antibodies of the present invention may also be used in methods for treating cancer. Antibodies of the present invention may be modified as an antibody drug conjugate (ADC) for such uses, including treatment of solid tumors. The ADCs generally comprise the following, each independently of the others, an antibody, including the antibodies of the present invention, a linker, and a cytotoxic or cytostatic agent. The antibody in the ADC complex is intended to target tumor cells, where the expression of the epitope that is bound by the antibody is elevated or is abundant and not target healthy (non-tumor cells), where the expression of the epitope that is bound by the antibody is low or absent. The ADC complex then delivers the cytotoxic or cytostatic agent to the tumor cells and thereby inducing cytotoxic or cytostatic effects on the tumor.

Other factors important in the selection of an antibody for an ADC complex that is useful for the treatment of cancers include the target antigen should be displayed on the surface of the tumor cell and is available to the circulated antibody. Further, the target antigen should possess internalization properties, especially upon binding of the antibody to the target antigen, as it will facilitate the uptake of the cytotoxic and/or cytostatic agent into the tumor cells which may enhance the cytotoxic effect of the ADC.

The cytotoxic and/or cytostatic agent may be any agent known to inhibit the growth and/or replication of and/or kill cells, and in particular cancer and/or tumor cells. The cytotoxic/cytostatic moiety used in ADCs should be of high stability in systemic circulation and in some cases, within cellular compartments such as lysosomes. Cytotoxic/cytostatic moieties that are useful for the methods and compositions of the present invention should have an in vitro IC50 in the nanomolar range, and even more preferably, in the sub-nanomolar range for cancer cell lines. In addition, a cytotoxic/cytostatic moiety used in ADCs should be sufficiently soluble in the aqueous environment for administration. Other considerations for cytotoxic/cytostatic moieties for use in ADCs include, but are not limited to low immunogenicity, small molecular weight and long half-life. The chemistry of the cytotoxic/cytostatic moiety should also allow for conjugation to the linker without interfering with the internalization or binding properties of the antibody and promoting its anti-tumor effects.

Numerous agents having cytotoxic and/or cytostatic properties are known in the literature. Any cytotoxic and/or cytostatic agent that include or may be modified to include a site of attachment to an antibody may be included in an ADC complex suitable for use as an anti-cancer or anti-tumor treatment. In general, they can be divided into two classes: microtubule disrupting agents and DNA-damaging agents. Non-limiting examples of classes of cytotoxic and/or cytostatic agents belonging to these two classes include radionuclides, alkylating agents, topoisomerase I inhibitors, topoisomerase II inhibitors, DNA intercalating agents, RNA/DNA antimetabolites, cell cycle modulators, kinase inhibitors, protein synthesis inhibitors, histone deacetylase inhibitors, mitochondria inhibitors and antimitotic agents.

Generally, microtubule disrupting agents can be divided into two classes: auristatin and maytansinoids. Auristatin is a synthetic anti-neoplastic agent derived from dolastatin 10. Dolastin 10 is not suitable for use as part of an ADC due to its non-specific cytotoxic effects, but synthetic analogues of dolastin 10 such as monomethyl auristatin E (MMAE) and monomethyl auristatin F (MMAF) are currently being used in ADC complexes. Both MMAE and MMAF work as cytotoxic/cytostatic agents by blocking tubulin polymerization, resulting in cell cycle arrest and apoptosis.

Maytansinoids are a second class of microtubule disrupting agent that can be used as the cytotoxic/cytostatic moiety in an ADC. Maytansinoids are isolated from the maytansine, a benzoansamacrolide. These drugs inhibit tubulin polymerization resulting in mitotic arrest and cell death. Derivatives of maytansine, mertansine (DM1) and ravtasine (DM4) have both been used as the cytotoxic/cytostatic moiety in ADCs.

DNA-damaging agents can also be used as the cytotoxic/cytostatic moiety in ADCs, including the methods and compositions of the present invention. DNA-damaging agents can be divided up into three main classes including calicheamicin, duocarmycin, and doxorubicin. Calicheamicins are a class of enediyne antitumor antibiotics derived from Micromonospora echinospora. Calicheamicin binds to the minor groove of DNA and stops DNA replication causing cell cycle arrest and cell death. Duocarmycin is a derivative extracted from Streptomyces strains of bacteria. Duocarmycin are another example of agents that bind to the minor groove of DNA and causes irreparable DNA alkylation, disrupting the nucleic acid architecture and structural integrity. Doxorubicin is a DNA intercalating agent that inhibits DNA synthesis, resulting in cell death.

One of skill in the art may pair specific classes of cytotoxic and/or cytostatic agents with certain types of tumors. As a non-limiting example, one of skill in the art may choose particularly effective cytotoxic agents from the DNA intercalating agent class or even a specific DNA intercalating agent if the targeted tumor has exhibited properties of multi-drug resistance.

Also included in an ADC complex is a linker whereby the cytotoxic and/or cytostatic agent is linked to the antibody. The linker linking a cytotoxic and/or cytostatic agent to the antibody of an ADC may be short, long, hydrophobic, hydrophilic, straight, flexible, or rigid, or may have segments comprised of one or more of the preceding properties. The linker may be linked to one or more sites on the antibody. Such linkages may be covalent. As can be appreciated by one of skill in the art, the linker can be directly covalently linked to the antibody and the cytotoxic and/or cytostatic agent or there can be a spacer between the linker and the antibody and/or a spacer between the linker and the cytotoxic and/or cytostatic agent. Linkers that are useful in ADCs need to be stable while in systemic circulation to avoid the release of the cytotoxic/cytostatic agent in order to minimize off-target effects. However, the linkers should possess the quality of being able to unleash the cytotoxic/cytostatic drug upon internalization into the target tumor cell after antibody binding. Linkers can be divided into cleavable and non-cleavage linkers.

Non-cleavable linkers consist of stable bonds that resist proteolytic degradation and provide higher stability than cleavable linkers. The mechanism of action of non-cleavable linkers is based on the internalization of the ADC after the antibody has bound to its target on the surface of the target tumor cell. After internalization, the ADC complex is then degraded in the lysosome of the target cell that results in the release of the cytotoxic/cytostatic agent inside the target cell to facilitate the tumor cell killing effects of the ADC. Because the non-cleavable linker is more stable, it minimizes the chances of releasing the cytotoxic/cytostatic agent at off-target sites and thus do not harm healthy cells or minimizing off-target effects.

Cleavable linkers are the second major class of linkers used as part of an ADC complex. The main feature of cleavage linkers is that they are sensitive and are cleaved as a result of environmental factors such as pH, specific lysosomal enzymes, etc. Different classes of cleavable linkers include but are not limited to acid-sensitive or acid-labile linkers, lysosomal protease-sensitive linkers, beta-glucuronide liners, and glutathione-sensitive disulfide linkers. Acid sensitive or acid-labile linkers are linkers that are stable at certain pH, usually at alkaline pH such as in systemic circulation and are sensitive to acidic environments such as lysosomal and/or endosomal acidic tumor microenvironments. Upon internalization within the target tumor cells, the linker gets hydrolyzed in its pH sensitive environment. Lysosomal protease-sensitive linkers are also known as peptide-based linkers. These linkers take advantage of the higher expression of some lysosomal proteases in tumor cells compared to non-tumor cells such as cathepsin B. Lysosomal protease-sensitive linkers such as cathepsin B sensitive linkers are selectively uptaken into tumor cells through receptor mediated endocytosis. Another advantage of lysosomal protease-sensitive linkers include stable in unsuitable pH environment and resistant to serum enzymes making them stable in systemic circulation and decreasing off target effects. An example of lysosomal protease sensitive liner is valine-citrulline (v-c). Beta-glucuronide linkers are another class of protease-sensitive linker that is recognized by and hydrolyzed by beta-glucuronidase for the cytotoxic/cytostatic agent release from the ADC complex. Lysosomes and tumor necrotic regions are high in beta-glucuronidase, the enzyme being inactive at physiologic pH and active at lysosomal pH. This selectivity allows for cleavage of the linker and thereby the release of the cytotoxic/cytostatic moiety at desired site and minimizing off target effects. Glutathione-sensitive disulfide linkers are another class of linkers that can be used in the methods and compositions of the present invention. Glutathione is a low molecular weight thiol that is found in intracellular compartments of cells. Glutathione is highly released during cell survival, tumor growth and cell stress conditions such as hypoxia, therefore higher concentrations of glutathione can be found in tumors compared to non-tumor tissues. Glutathione-sensitive linkers are stable in systemic circulation and are selectively cleaved when targeted to the tumor by the ADC by the higher concentration of glutathione.

In an embodiment, the linker and cytotoxic agent as part of an ADC complex comprise DM1, a microtubule-disrupting agent and a linker, succinimidyl-4-(N-maleimidomethyl)cyclohexane-1-carboxylate (SMCC). Such linker and cytotoxic agent can be used to form an ADC complex with antibodies of the present invention.

Linkers can be chemically stable to conditions outside the cell and may be designed to cleave, degrade, and/or otherwise detach once inside the cell. Other ADCs may not need to be internalized by the targeted tumor cell and achieve its cytotoxic and/or cytostatic effects on the surface of the tumor cell after the antibody of the ADC complex has bound to its epitope. A wide variety of linkers useful to linking drugs, including cytotoxic and/or cytostatic agents, to antibodies in the context of ADCs are known in the art. Any of these linkers, as well as other linkers, may be used to link cytotoxic and/or cytostatic agents to the antibodies of the present disclosure to form ADC complexes that are useful in the treatment of cancer or target tumor cells.

ADCs that are part of the present invention may synthesized using methods known in the art. The specific chemistry of linking the antibody to the linker and also to the cytotoxic and/or cytostatic agent will depend upon, among other things, the identity of the cytotoxic and/or cytostatic agent, the linker and any spacer that is attached to the antibody. Generally, the chemistry used to link the linker to the antibody and the cytotoxic and/or cytostatic agent should not alter the integrity of the antibody, for example, its ability to bind to its target. Preferably, the binding properties of the conjugated antibody will closely resemble those of the unconjugated antibody. A variety of chemistries and techniques for conjugating molecules to biological molecules such as antibodies are well-known in the art.

ADCs of the present invention may be useful for the treatment of cancers or tumors where the epitope targeted by the antibody is highly expressed or upregulated on the cancer or tumor cells and not expressed or expressed at a low level in healthy cells. In such cases, the targeted tumor or cancer cell can be part of the primary tumor or in other cases, the tumor or cancer cell can be part of a metastasis. In some embodiments, the targeted tumor is a solid tumor. In some embodiments the targeted tumor is colon adenocarcinoma, small intestine tumors, or rectal tumors. In some embodiments the targeted cancer or tumor is a colon carcinoma, a colon adenocarcinoma, a small intestine cancer, a small intestine adenocarcinoma, a skin squamous carcinoma, a rectal cancer, a thyroid cancer, a lung cancer, an esophagus cancer, a colon cancer, an ovarian cancer, a prostate cancer, a liver cancer, a pancreatic cancer, a uterine cancer, a bladder cancer, a stomach cancer, a cervical cancer, a gallbladder cancer, an endometrial cancer, or a breast cancer. In some embodiments, the breast cancer is an invasive ductal or invasive lobular carcinoma. In some embodiments, the esophagus cancer is an adenocarcinoma or a squamous cell carcinoma. In some embodiments, the liver cancer is a cholangiocellular carcinoma. In some embodiments, the ovarian cancer is a mucinous adenocarcinoma. In some embodiments, the rectal cancer is an adenocarcinoma or a rectal cancer lymph node metastasis. In some embodiments, the stomach cancer is an adenocarcinoma. In some embodiments, the cervical cancer is a cervix squamous cell carcinoma. In some embodiments, the endometrial cancer is an endometrioid adenocarcinoma. In some embodiments, the small intestine cancer is a small intestine cancer lymph node metastasis. In some embodiments, the gallbladder cancer is an adenocarcinoma.

Antibodies of the present invention may also be used in methods of aiding diagnosis of disease, such as diagnosis of cancer in an individual. Such cancers include cancers of the digestive system such as pancreatic or colon cancer. Such methods include using the antibodies of the present invention to determine the level of sLeA and/or sLeC binding in an individual or specific tissue in an individual. As used herein, methods for “aiding diagnosis” means that these methods assist in making a clinical determination regarding the classification, or nature, or cancer, and may or may not be conclusive with respect to the definitive diagnosis. Accordingly, a method of aiding diagnosis of cancer can comprise the step of detecting the level of sLeA and/or sLeC in a biological sample from the individual and/or determining the level of sLeA and/or sLeC in the sample. Antibodies recognizing the epitope or a portion thereof may also be used to create diagnostic immunoassays for detecting antigenic determinant released in the bodily fluids, including but not limited to blood, saliva, urine, pulmonary fluid, or ascites fluid. Similarly, such immunoassays using the antibodies of the present invention may be used to monitor the efficacy of treatment and/or disease remission. In such instances, the presence and/or levels of the antigenic determinant reactive to the antibody of the present invention may be useful as a biomarker in order to monitor disease activity and/or progression.

In some embodiments, an antibody of the present disclosure may also be used in a method of detecting the presence and/or measuring the level of sLeA and/or sLeC in an individual or specific tissue in an individual.

In some embodiments, the antibodies binding to sLeA of the present invention may be used alone for the treatment or ameliorating one or more disease symptom. In other embodiments, the antibodies of the present invention may be used in combination with other therapeutics or pharmaceutical agents to treat or ameliorate one or more disease symptom.

In some embodiments, an antibody of the present disclosure may also be used in a method of detecting the presence and/or measuring the level of sLeA and/or sLeC in an individual or specific tissue in an individual.

Certain aspects of the present disclosure relate to polynucleotides (e.g., isolated polynucleotides) and/or vectors (e.g., expression vectors) encoding an antibody of the present disclosure, as well as host cells (e.g., isolated host cells) comprising the polynucleotides or vectors. In some embodiments, the host cell is a prokaryotic host cell, such as a bacterial host cell (e.g., E. coli). Suitable prokaryotic host cells include without limitation eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, etc. In some embodiments, the host cell is a eukaryotic host cell such as yeast, fungi, insect, plant, animal, human, or nucleated cells from other multicellular organisms. For example, filamentous fungi or yeast are suitable cloning or expression hosts for antibody-encoding vectors. Saccharomyces cerevisiae, or common baker's yeast, is the most commonly used among lower eukaryotic host microorganisms. Suitable host cells for the expression of glycosylated antibody are also derived from multicellular organisms (invertebrates and vertebrates). Examples of invertebrate cells include plant and insect cells. Examples of vertebrate or mammalian cells include, e.g., monkey kidney CVI line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; a human hepatoma line (Hep G2); Chinese hamster ovary (CHO) cells, including DHFR″ CHO cells (Urlaub et al., Proc. Natl. Acad. Sci. USA 77:4216 (1980)); and myeloma cell lines such as NSO and Sp2/0.

Other aspects of the present disclosure relate to methods of production using a host cell of the present disclosure. Antibodies or antigen-binding portions thereof can be produced using recombinant methods. For recombinant production of an antibody or antigen-binding portion, nucleic acid encoding the antibody/portion is isolated and inserted into a vector for further cloning (amplification of the DNA) or for expression. DNA encoding the antibody can be isolated and sequenced using conventional procedures (e.g., by using oligonucleotide probes that are capable of binding specifically to genes encoding the heavy and light chains of the antibody). The vector components generally include, but are not limited to, one or more of the following: a signal sequence, an origin of replication, one or more marker genes, an enhancer element, a promoter, and a transcription termination sequence.

Various formulations of antibodies of the present invention or fragments thereof may be used for administration. In some embodiments, the antibodies of the present invention or fragments thereof may be administered neat. In addition to the pharmacologically active agent, the compositions of the present invention may contain suitable pharmaceutically acceptable carriers comprising excipients and auxiliaries that are well known in the art and are relatively inert substances that facilitate administration of a pharmacologically effective substance or which facilitate processing of the active compounds into preparations that can be used pharmaceutically for delivery to the site of action. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers.

Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington, The Science and Practice of Pharmacy 20th Ed. Mack Publishing (2000).

Suitable formulations for oral administration include hard or soft gelatin capsules, pills, tablets, including coated tablets, elixirs, suspensions, syrups or inhalations and controlled release forms thereof. Additionally, oral administration of a peptide or protein therapeutic such as an antibody therapeutic can be coated with an active ingredient or formulated to be resistant against digestion in the stomach. Oral administration may also include sublingual and buccal administration.

Generally, pharmaceutical agents may be formulated for administration by injection (e.g., intraperitoneally, intravenously, subcutaneously, intramuscularly, etc.), although other forms of parenteral administration (e.g., local administration, topical, intranasal, intrapulmonary, ocular, and rectal) can be also used. Accordingly, antibodies of the present invention are preferably combined with pharmaceutically acceptable vehicles such as saline, Ringer's solution, dextrose solution, and the like. The pharmaceutical composition may be administered using any device that may help migration or deposition of an active component to a target cell.

The particular dosage regimen, i.e., dose, timing and repetition, will depend on the particular individual and that individual's medical history and mode of administration. One of ordinary skill in the art may determine a dose/dosing regimen that is suitable. Generally, a dose of at least about 0.1 mg/kg body weight, or more preferably, at least about 1 mg/kg body weight, or at least about 5 mg/kg body weight, even more preferably at least about 10 mg/kg body weight or at least about 20 mg/kg body weight is administered by injection.

In some embodiments, one or more doses of the antibodies of the present invention or fragments thereof will be administered during a course of treatment. Empirical considerations, such as half-life, generally will contribute to the determination of the dosage. Antibodies, which are compatible with the human immune system, such as humanized antibodies or fully human antibodies ma y be used to prolong half-life of the antibody and prevent the antibody being attacked by the host's immune system. Frequency of administration may be determined and adjusted over the course of therapy, and may be based on the reduction of one of more clinical symptoms of the disease. Alternatively, sustained continuous release formulations of the antibodies of the present invention may be appropriate. Various formulations and devices for achieving sustained release are known in the art.

Certain aspects of the present disclosure further relate to kits or articles of manufacture that comprise an ADC of the present disclosure. Optionally, the kits described herein may contain one or more pharmaceutically acceptable carriers, such as the exemplary carriers described herein. In some embodiments, a kit of the present disclosure includes a pharmaceutical composition of the present disclosure. Kits described herein may find use, e.g., in the methods or uses of the present disclosure.

Kits may optionally provide additional components such as buffers and interpretive information. Normally, the kit comprises a container and a label or package insert(s) on or associated with the container. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

In some embodiments, the kits further include a package insert comprising instructions for administration of an ADC of the present disclosure to treat a cancer. In some embodiments, the cancer is a solid tumor. In some embodiments the cancer is colon adenocarcinoma, small intestine tumors, or rectal tumors. In some embodiments the cancer is a colon carcinoma, a colon adenocarcinoma, a small intestine cancer, a small intestine adenocarcinoma, a skin squamous carcinoma, a rectal cancer, a thyroid cancer, a lung cancer, an esophagus cancer, a colon cancer, an ovarian cancer, a prostate cancer, a liver cancer, a pancreatic cancer, a uterine cancer, a bladder cancer, a stomach cancer, a cervical cancer, a gallbladder cancer, an endometrial cancer, or a breast cancer. In some embodiments, the breast cancer is an invasive ductal or invasive lobular carcinoma. In some embodiments, the esophagus cancer is an adenocarcinoma or a squamous cell carcinoma. In some embodiments, the liver cancer is a cholangiocellular carcinoma. In some embodiments, the ovarian cancer is a mucinous adenocarcinoma. In some embodiments, the rectal cancer is an adenocarcinoma or a rectal cancer lymph node metastasis. In some embodiments, the stomach cancer is an adenocarcinoma. In some embodiments, the cervical cancer is a cervix squamous cell carcinoma. In some embodiments, the endometrial cancer is an endometrioid adenocarcinoma. In some embodiments, the small intestine cancer is a small intestine cancer lymph node metastasis. In some embodiments, the gallbladder cancer is an adenocarcinoma.

The kits of the present disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer), or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an ADC described herein. The container may further comprise a second pharmaceutically active agent. In some embodiments, a kit may further include any other material or device useful in a treatment, including without limitation one or more containers, tubing, sterilizing agents or equipment, cannulae, syringes, and the like.

The following examples are provided to illustrate, but not to limit the invention.

### Examples

### Example 1. Generation of Humanized Antibody Clones

Humanized antibodies, with a heavy chain variable region comprising one of the heavy chain variable regions from Table 1 above and a light chain variable region comprising one of the light chain variable regions from Table 2 above were synthesized and incorporated into an IgG1 backbone. Heavy and light chain variable region pairs that were incorporated into an IgG1 backbone and synthesized were the ones in the Table 6 below.

TABLE 6
Humanized Antibody Clones

| Humanized | Heavy Chain | | Light Chain | |
|---|---|---|---|---|
| Antibody | Variable | SEQ ID | Variable | SEQ ID |
| Clone | Region | NO: | Region | NO: |
| 4764 | HvCh1 | 1 | LiCh1 | 5 |
| 4765 | HvCh2 | 2 | LiCh1 | 5 |
| 4766 | HvCh2 | 2 | LiCh2 | 6 |
| 4767 | HvCh2 | 2 | LiCh3 | 7 |
| 4768 | HvCh2 | 2 | LiCh4 | 8 |
| 4769 | HvCh3 | 3 | LiCh1 | 5 |
| 4770 | HvCh3 | 3 | LiCh2 | 6 |
| 4771 | HvCh3 | 3 | LiCh3 | 7 |
| 4772 | HvCh3 | 3 | LiCh4 | 8 |

Humanized antibodies with the above combinations of heavy and light chain variable region pairs were produced by transfecting the plasmids into mammalian cells and purified using affinity chromatography.

Of the nine humanized antibody clones in Table 6, two antibody clones (4764 and 4767) showed weak expression, resulting in less than 1 mg of antibody after purification. The production levels for these two clones were deemed too low for practical production cell line optimization. The remaining seven clones were then screened for binding affinity to sLeA, sLeC and sLeX.

### Example 2. Binding Affinity to Sialyl Lewis A (sLeA) and Sialyl Lewis C (sLeC)

An in vitro evaluation of the binding affinity to sLeA and sLeC of the humanized antibodies from Example 1 were performed using a Biocore T100 SPR biosensor using a CM7 sensor chip with PBS-p (0.005% Tween-20) running buffer at 25 degrees Celsius. The antibodies were coupled to ˜30,000 RU at 50 ug/ml in 10 mM NaAcetate pH 5.0 over an NHS/EDC activated surface. A reference surface was activated and blocked to serve as a control. Carbohydrate samples (sLeA, sLeC and sialyl Lewis X (all from Dextra Labs)) were dissolved in PBS-p running buffer up to a stock concentration of 10 mM. Each was then prepared in a 2-fold dilution series up to 300 μM and each concentration series was tested over the antibody surfaces. Response data were processed by subtracting the responses from the reference surfaces as well as a buffer injection. Binding constants were determined at 25 degrees Celsius.

We tested a murine antibody (GM35) described in Brazil, J Immunol 191:4804-4817 (2013) for binding affinities for sLeA, sLeC and sLeX using the methods described above. The results for this antibody did not show binding to sLeX, had a K_{D} for sLeA of 41.5 μM to 46.8 μM, and a K_{D} for sLeC of 68.8 μM to 78.2 μM. Comparatively, another antibody that was tested was NS19-9 (Dako, Carpenteria, CA), an antibody known to bind to sLeA, which did not show binding to sLeX, had a K_{D} for sLeA of 39.5 μM, and a K_{D} for sLeC of 1.5 mM.

### Example 3. Binding Affinity to Sialyl Lewis a (sLeA) and Sialyl Lewis C (sLeC) for Humanized Antibody Clones

Seven of the humanized antibody clones from Example 1 were tested for binding affinity to sLeA, sLeC and sLeX. Methods used were similar to those described in Example 2 above, except with sLeX only at one concentration of 300 μM. Mean binding K_{D} for sLeA and sLeC for each of the seven humanized antibody clones are shown in Table 7 below.

TABLE 7
Binding Affinity for Humanized Antibody Clones

| Humanized | | |
|---|---|---|
| Antibody | sLeA Mean K_{D} | sLeC Mean K_{D} |
| Clone | in μM | in μM |
| 4765 | 731.7 | 306.3 |
| 4766 | 620.7 | 538.0 |
| 4768 | 552.0 | 460.0 |
| 4769 | 81.3 | 103.7 |
| 4770 | 69.1 | 93.2 |
| 4771 | 59.2 | 85.4 |
| 4772 | 48.3 | 77.1 |

None of the above antibody clones showed binding to sLeX in three independent runs at a concentration of 300 μM of the carbohydrate. From the above binding affinity data, humanized antibody clones with HvCh3 variable regions had the highest affinity for both sLeA and sLeC.

### Example 4. Detection of Clone 4772 Epitope Expression on T84 and U87-MG Cells

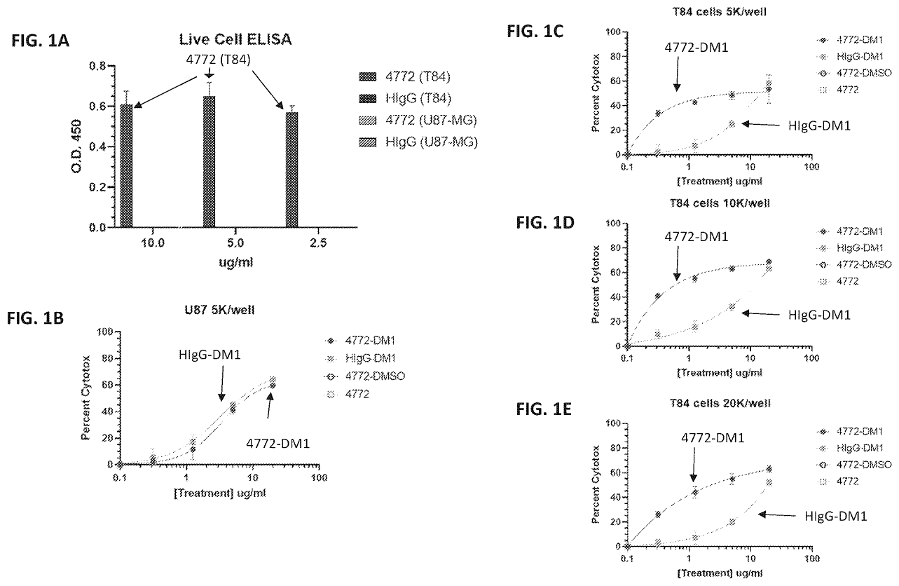

Live cell ELISA were performed on the cells at the 72-hour time point to confirm binding of the clone 4772 and hIgG antibodies on T84 (colon carcinoma derived from lung metastasis) and U87-MG (glioblastoma) cells. Briefly, cells were plated onto tissue culture treated 96-well plates at the desired cell density at 100 μl per well in F12/DMEM media with 5% FBS, 24 hours prior to the start of the experiment. Prior to the start of the experiment, the plates were checked microscopically to ensure proper cell attachment. Media was removed from the plates and 100 μl Hank's Balanced Salt Solution with Calcium and Magnesium (HBSS, VWR, Pennsylvania) containing 1% (w/v) bovine serum albumin (BSA, VWR, Pennsylvania) and 0.01% (v/v) sodium azide (NaN3, VWR, Pennsylvania) containing the appropriate antibody at the desired concentration was then added to the appropriate wells and incubated at room temperature for 1 hour. The plates were then washed three times with HBSS at 200 μl volume per wash. The plates were tapped dry and 100 μl of the appropriate horse radish peroxidase (HRP)-conjugated, species-specific antibody (Jackson ImmunoResearch, Pennsylvania) at the appropriate concentration in HBSS was added to each well. The plates were allowed to incubate for 30 minutes at room temperature. The plates were then washed three times with HBSS and tapped dry. 100 μl of TMB substrate (KPL Seracare, MA) was added to each well and allowed for color development. TMB Stop Solution (KPL Seracare, MA) was added to each well at a volume of 100 μl per well and the plates were read at O.D. 450 nM on a GloMax Plate Reader (Promega, WI) using the absorbance setting. FIG. 1A shows the results of the live cell ELISA results on T84 and U87-MG cells. Clone 4772 bound to T84 cells, while no appreciable binding was detected on U87-MG cells or in the hIgG control conditions. Similar experiments were also performed on A431 (skin squamous carcinoma), A549 (lung adenocarcinoma), and Colo-205 (colon adenocarcinoma) cells. Antibody clone 4772 bound to A431 and Colo-205 cells, while no appreciable binding was detected on A549 cells or in the hIgG control conditions.

### Example 5. In Vitro Assay for Cytotoxicity

Clone 4772 was selected for testing for in vitro cell toxicity potential as an antibody drug conjugate (ADC). 1 mg of clone 4772 or human IgG1 (Athens Research and Technology, GA) in PBS was mixed with SMCC-DM1 (10 mM in DMSO, MedChemExpress, NJ) at a molar ratio of 1:10 (antibody: SMCC-DM1) and allowed to incubate overnight at room temperature. 1 mg of clone 4772, with equivolume of DMSO was also allowed to incubate overnight at room temperature to create a DMSO control (no ADC control). At the end of the incubation, buffer exchange was performed using Vivaspin-2 50,000 molecular weight cut-off concentrators (Sartorius, Germany). 20 volumes of PBS were used for the buffer exchange using manufacture's recommended conditions. Post buffer exchange, solutions of 4772-DM1, hIgG-DM-1, and 4772-DMSO were collected and each volume were adjusted to 1 ml in PBS.

T84 cells, a human carcinoma cell line derived from a lung metastasis of a colon carcinoma, were shown previously to support clone 4772 binding. Briefly described, T84 cells were plated onto 96-well tissue culture plates at cell densities of 5000 cells/well, 10,000 cells/well and 20,000 cells/well at a volume of 100 μl/well in F12/DMEM media with 5% fetal bovine serum (FBS). 24 hours after plating, the plates were checked microscopically to ensure proper cell attachment. 100 μl of 4772 only, 4772-DMSO, 4772-DM1, and hIgG-DM1 as 2×final concentration was added to the T84 cells. At the end of seventy-two hours after adding the antibodies, cell toxicities were measured using Cell Titer Glo (Promega, Wisconsin) according to manufacturer's recommended conditions. Luminescence units were read using a Promega GloMax plate reader using standard luminescence settings. Similar experiments using U87-MG cells were also performed. U87-MG cells are a human glioblastoma cell line that are epithelial in morphology.

FIG. 1B shows the cytotoxicity results from U87-MG cells (at 5000 cells/well). As expected, based on the lack of binding of clone 4772 to U87-MG cells in the live cell ELISA experiments, there were no differences detected in cytotoxicity between the clone 4772-DM1 conditions and the hIgG-DM1control conditions. FIGS. 1C, 1D, and 1E shows cytotoxicity results from T84 cells at 5000 cells/well, 10,000 cells/well, and 20,000 cells/well, respectively. Increased cell toxicity can be seen with increased concentrations of 4772-DM1 conditions and non-specific (hIgG-DM1) cytotoxicity was also observed at higher concentrations. Maximal cytotoxicity for 4772-DM1 conditions was observed at about 60% for all three cell densities.

Similar experiments for cytotoxicity were also performed on A431, A549, and Colo-205 cells. Sensitivity to 4772-DM1 was seen in A431 and Colo-205 cells and not A549 cells, which were shown in Example 4 above not to support binding of clone 4772 by live cell ELISA experiments.

### Example 6. Clone 4772 Epitope Expression on Cancer and Normal Tissue by Immunohistochemistry

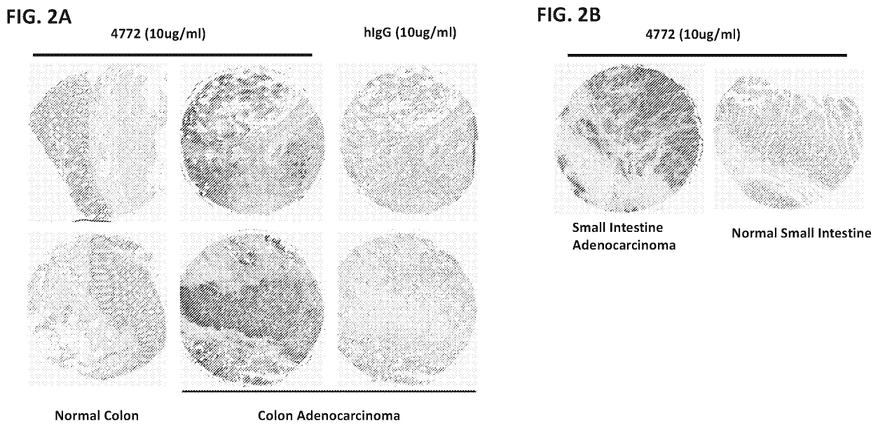

Tissue binding of antibody clone 4772 was tested in tissue microarrays for human normal and cancer tissues. The tissue microarrays (US BioMax, MD, USA) were prepared according to manufacturer's recommendations and stained using a Leica BOND RX staining platform (Leica Biosystems, IL, USA). 4772 was used at 10 ug/ml and rabbit anti-human secondary (AbCam, MA, USA) was used at 5 ug/ml in conjunction with the BOND Polymer Detection Kit (Leica Biosystems, IL, USA). As shown in FIGS. 2A and 2B, clone 4772 bound strongly to colon adenocarcinoma and small intestine adenocarcinoma tissue but not to normal colon or normal small intestine tissue. Additionally, hIgG control also did not stain colon adenocarcinoma (FIG. 2A) or small intestine adenocarcinoma tissues (data not shown).

Additional cancer tissue microarrays were also stained using clone 4772 and human IgG as a negative control. Strong staining was observed in the following cancer tissue types: thyroid, lung, esophagus, colon, rectum, ovarian, prostate, liver, pancreas, uterine, bladder, stomach, cervix and breast. There was no appreciable staining detected in the human IgG stained samples.

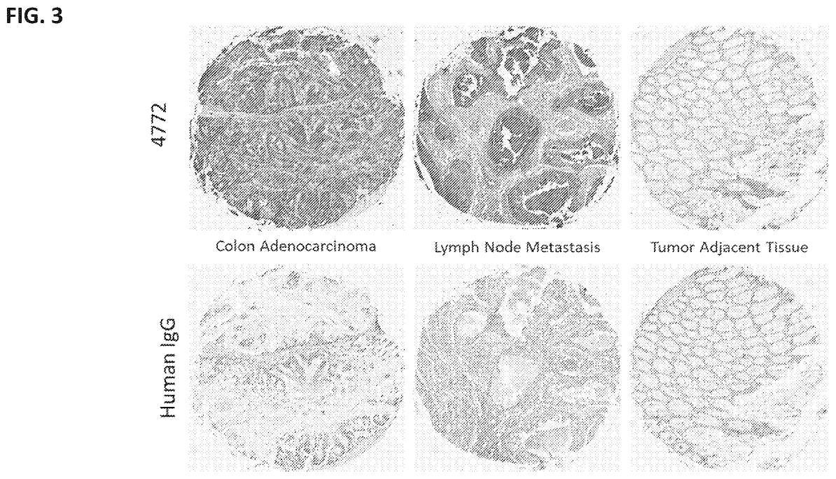

We also examined the tissue binding of antibody clone 4772 on colon adenocarcinoma, matched lymph node metastasis of the colon adenocarcinoma and matched tumor adjacent colon tissue. As shown in FIG. 3, clone 4772 bound strongly to colon adenocarcinoma tissue and the matched lymph node metastasis tissue, while it did not show staining in the matched tumor adjacent colon tissue. There was no appreciable staining detected in the human IgG stained samples. Staining was also performed on various other solid tumors, and the results are summarized in Table 8, below.

TABLE 8
Clone 4772 Epitope Expression on Human Solid Tumors

| Cancer Type | # Samples | # Positive | % Positive |
|---|---|---|---|
| Bladder | 14 | 13 | 92% |
| Breast (Invasive Ductal and | 22 | 11 | 50% |
| Invasive Lobular Carcinoma) | | | |
| Cervix | 8 | 4 | 50% |
| Esophagus (Adenocarcinoma and | 161 | 73 | 45% |
| Squamous Cell Carcinoma) | | | |
| Gallbladder, Adenocarcinoma | 5 | 5 | 100% |
| Liver (All Types) | 40 | 20 | 50% |
| Liver, Cholangiocellular Carcinoma | 19 | 16 | 84% |
| Lung Cancer | 20 | 13 | 65% |
| Ovarian (All Types) | 25 | 10 | 40% |
| Ovarian, Mucinous Adenocarcinoma | 4 | 4 | 100% |
| Pancreas (All Types) | 20 | 17 | 85% |
| Rectum, Adenocarcinoma and | 19 | 14 | 73% |
| Lymph Node Metastasis | | | |
| Small Intestine (All types | 54 | 38 | 70% |
| and Lymph Node Metastasis) | | | |
| Stomach, Adenocarcinoma | 11 | 7 | 63% |
| Uterus, Cervix Squamous Cell | 14 | 11 | 78% |
| Carcinoma and Endometrioid | | | |
| Adenocarcinoma | | | |

SEQUENCES
Amino acid sequence of HvCh1
SEQ ID NO: 1
EVQLVESGGGLVQPGGSLRLSCAASGFTFSTNAMSWVRQAPGKGLEWVSR
LRPKSDNYATYYADSVKGRFTISRDNSKNTLYLQMNSLRAEDTAVYYCAK
GTGFWGQGTTVTVSS;
Amino acid sequence of HvCh2
SEQ ID NO: 2
EVQLVESGGGLVQPGGSLRLSCAASGFTFSTNAMSWVRQAPGKGLEWVAR
LRPKSDNYATYYADSVKGRFTISRDNSKNTLYLQMNSLRAEDTAVYYCVT
GTGFWGQGTTLTVSS;
Amino acid sequence of HvCh3
SEQ ID NO: 3
EVQLVESGGGLVQPGGSLRLSCAASGFTFSTNAMSWVRQAPGKGLEWVAR
LRPKSDNYATYYADSVKGRFTISRDDSKNTLYLQMNSLRAEDTAVYYCVT
GTGFWGQGTTLTVSS;
Amino acid sequence of HvCh4
SEQ ID NO: 4
EVQLVESGGGLVQPGGSLRLSCAASGFTFSTNAMSWVRQAPGKGLEWVAR
LRPKSDNYATYYADSVKGRFTISRDDSTSTLYLQMNSLRAEDTAVYYCVT
GTGFWGQGTTLTVSS;
Amino acid sequence of LiCh1
SEQ ID NO: 5
DIVMTQSPDSLAVSLGERATINCKSSQSLLNSGNQKNYLTWYQQKPGQPP
KLLIYWTSTRESGVPDRFSGSGSGTDFTLTISSLQAEDVAVYYCONDYTS
PYTFGQGTKLEIK;
Amino acid sequence of LiCh2
SEQ ID NO: 6
DIVMTQSPDSLAVSLGERVTMNCKSSQSLLNSGNQKNYLTWYQQKPGQPP
KLLIYWTSTRESGVPDRFSGSGSGTDFTLTISSVQAEDVAVYYCONDYTS
PYTFGQGTKLEIK;
Amino acid sequence of LiCh3
SEQ ID NO: 7
DIVMTQSPDSLAVSLGERATINCKSSQSLLNSGNQKNYLTWYQQKPGQPP
KLLFYWTSTRESGVPDRFSGSGSGTDFTLTISSLQAEDVAVYYCONDYTS
PYTFGQGTKLEIK;
Amino acid sequence of LiCh4
SEQ ID NO: 8
DIVMTQSPDSLAVSLGERVTMNCKSSQSLLNSGNQKNYLTWYQQKPGQPP
KLLFYWTSTRESGVPDRFSGSGSGTDFTLTISSVQAEDVAVYYCQNDYTS
PYTFGQGTKLEIK;
Amino acid sequence of LiCh5
SEQ ID NO: 9
DIVMTQSPDSLAVSLGERVTMNCKSSQSLLNSGNQKNYLTWYQQKPGQPP
KLLFYWTSTRESGVPDRFSGSGSGTDFTLTISSVQAEDLAVYYCONDYTS
PYTFGQGTKLEIK;
Amino acid sequence of LiCh6
SEQ ID NO: 10
DIVMTQSPDSLAVSLGERVTMNCKSSQSLLQSGNQKNYLTWYQQKPGQPP
KLLFYWTSTRESGVPDRFSGSGSGTDFTLTISSVQAEDLAVYYCONDYTS
PYTFGQGTKLEIK;
Amino acid sequence of LiCh7
SEQ ID NO: 11
DIVMTQSPDSLAVSLGERVTMNCKSSQSLLSSGNQKNYLTWYQQKPGQPP
KLLFYWTSTRESGVPDRFSGSGSGTDFTLTISSVQAEDLAVYYCONDYTS
PYTFGQGTKLEIK;
Amino acid sequence of heavy chain CDR-1
SEQ ID NO: 12
GFTFSTNAMS;
Amino acid sequence of heavy chain CDR-2
SEQ ID NO: 13
RLRPKSDNYATY;
Amino acid sequence of heavy chain CDR-3
SEQ ID NO: 14
VTGTGF;
Amino acid sequence of light chain CDR-1
SEQ ID NO: 15
KSSQSLLNSGNQKNYLT;
Amino acid sequence of light chain CDR-1B
SEQ ID NO: 16
KSSQSLLQSGNQKNYLT;
Amino acid sequence of light chain CDR-1C
SEQ ID NO: 17
KSSQSLLSSGNQKNYLT;
Amino acid sequence of light chain CDR-2
SEQ ID NO: 18
WTSTRES;
Amino acid sequence of light chain CDR-3
SEQ ID NO: 19
QNDYTSPYT;
Amino acid sequence of heavy chain CDR-3B
SEQ ID NO: 20
AKGTGF;

What is claimed is:

1. An antibody drug conjugate (ADC) comprising:
a. an antibody comprising a heavy chain variable region and a light chain variable region, wherein the heavy chain variable region comprises a CDR-H1 comprising the sequence of SEQ ID NO:12, a CDR-H2 comprising the sequence of SEQ ID NO: 13, and a CDR-H3 comprising the sequence of SEQ ID NO: 14; and wherein the light chain variable region comprises a CDR-L1 comprising a sequence selected from the group consisting of SEQ ID NOs: 15-17, a CDR-L2 comprising the sequence of SEQ ID NO: 18, and a CDR-L3 comprising the sequence of SEQ ID NO:19;
b. a linker; and
c. a cytotoxic or cytostatic agent;
wherein the cytotoxic or cytostatic agent is linked to the antibody via the linker.

2. The ADC of claim 1, wherein:
(a) the heavy chain variable region comprises a CDR-H1 comprising the sequence of SEQ ID NO: 12, a CDR-H2 comprising the sequence of SEQ ID NO: 13, and a CDR-H3 comprising the sequence of SEQ ID NO:14; and the light chain variable region comprises a CDR-L1 comprising the sequence of SEQ ID NO: 15, a CDR-L2 comprising the sequence of SEQ ID NO:18, and a CDR-L3 comprising the sequence of SEQ ID NO:19;
(b) the heavy chain variable region comprises a CDR-H1 comprising the sequence of SEQ ID NO:12, a CDR-H2 comprising the sequence of SEQ ID NO:13, and a CDR-H3 comprising the sequence of SEQ ID NO: 14; and the light chain variable region comprises a CDR-L1 comprising the sequence of SEQ ID NO:16, a CDR-L2 comprising the sequence of SEQ ID NO: 18, and a CDR-L3 comprising the sequence of SEQ ID NO: 19; or
(c) the heavy chain variable region comprises a CDR-H1 comprising the sequence of SEQ ID NO: 12, a CDR-H2 comprising the sequence of SEQ ID NO:13, and a CDR-H3 comprising the sequence of SEQ ID NO: 14; and the light chain variable region comprises a CDR-L1 comprising the sequence of SEQ ID NO: 17, a CDR-L2 comprising the sequence of SEQ ID NO: 18, and a CDR-L3 comprising the sequence of SEQ ID NO: 19.

3. The ADC of claim 1 or claim 2, wherein the antibody comprises a heavy chain variable region comprising a sequence selected from the group consisting of SEQ ID NOs: 2-4, and a light chain variable region comprising a sequence selected from the group consisting of SEQ ID NOs: 5-11.

4. The ADC of any one of claims 1-3, wherein the antibody binds to sialyl Lewis A (sLeA) and sialyl Lewis C (sLeC).

5. The ADC of any one of claims 1-4, wherein the antibody does not bind to sialyl Lewis X (sLeX).

6. The ADC of claim 4 or claim 5, wherein the binding affinity of the antibody to sLeA is of K_{D} of about 100 μM or less, and the binding affinity of the antibody to sLeC is of K_{D} of about 110 μM or less.

7. The ADC of any one of claims 1-6, wherein the antibody is a humanized antibody.

8. The ADC of any one of claims 1-7, wherein the antibody is of the IgA, IgD, IgE, IgG, or IgM class.

9. The ADC of claim 8, wherein the antibody is of the IgG class and has an IgG1, IgG2, IgG3, or IgG4 isotype.

10. The ADC of claim 9, wherein the antibody is of the IgG class and has a human IgG1 isotype.

11. The ADC of any one of claims 1-10, wherein the antibody is an antibody fragment comprising an antigen binding portion.

12. The ADC of claim 11, wherein the antibody fragment is a scFv, (scFv)₂, Fab, Fab′, or F(ab′)₂ fragment.

13. The ADC of any one of claims 1-12, wherein the linker is a cleavable linker or a non-cleavage linker.

14. The ADC of claim 13, wherein the linker is a valine-citrulline (v-c) linker, a beta-glucuronide linker, or a succinimidyl-4-(N-maleimidomethyl cyclohexane)-1-carboxylate (SMCC) linker.

15. The ADC of any one of claims 1-14, wherein the cytotoxic or cytostatic agent is a microtubule-disrupting agent or a DNA-damaging agent.

16. The ADC of claim 15, wherein the cytotoxic or cytostatic agent is a radionuclide, an alkylating agent, a topoisomerase I inhibitor, a topoisomerase II inhibitor, a DNA intercalating agent, a RNA/DNA antimetabolite, a cell cycle modulator, a kinase inhibitor, a protein synthesis inhibitor, a histone deacetylase inhibitor, a mitochondria inhibitor, or an antimitotic agent.

17. The ADC of claim 15, wherein the cytotoxic or cytostatic agent is a microtubule-disrupting agent selected from the group consisting of an auristatin and a maytansinoid.

18. The ADC of claim 17, wherein the auristatin is an analogue of dolastin 10.

19. The ADC of claim 18, wherein the analogue of dolastin 10 is monomethyl auristatin E (MMAE) or monomethyl auristatin F (MMAF).

20. The ADC of claim 17, wherein the maytansinoid is mertansine (DM1) or ravtasine (DM4).

21. The ADC of claim 15, wherein the cytotoxic or cytostatic agent is a DNA-damaging agent selected from the group consisting of a calicheamicin, duocarmycin, and doxorubicin.

22. The ADC of any one of claims 1-16 and 20, wherein the linker is an SMCC linker and the cytotoxic or cytostatic agent is DM1.

23. The ADC of any one of claims 1-22, wherein the ADC further comprises a spacer between the linker and the antibody, and/or a spacer between the linker and the cytotoxic or cytostatic agent.

24. A method of treating cancer, comprising administering to a patient in need thereof an effective amount of the ADC of any one of claims 1-23.

25. The method of claim 24, wherein the cancer is a solid tumor.

26. The method of claim 24 or claim 25, wherein the cancer is a colon carcinoma, a colon adenocarcinoma, a small intestine cancer, a small intestine adenocarcinoma, a skin squamous carcinoma, a rectal cancer, a thyroid cancer, a lung cancer, an esophagus cancer, a colon cancer, an ovarian cancer, a prostate cancer, a liver cancer, a pancreatic cancer, a uterine cancer, a bladder cancer, a stomach cancer, a cervical cancer, a gallbladder cancer, an endometrial cancer, or a breast cancer.

27. A pharmaceutical composition comprising the ADC of any one of claims 1-23 and a pharmaceutically acceptable carrier.

28. A kit comprising the ADC of any one of claims 1-23 and an optional pharmaceutically acceptable carrier.

29. The kit of claim 28, wherein the kit further comprises a package insert comprising instructions for administration of the ADC to treat a cancer in a patient in need thereof.

30. The kit of claim 29, wherein the cancer is a solid tumor.

31. The kit of claim 29 or claim 30, wherein the cancer is a colon carcinoma, a colon adenocarcinoma, a small intestine cancer, a small intestine adenocarcinoma, a skin squamous carcinoma, a rectal cancer, a thyroid cancer, a lung cancer, an esophagus cancer, a colon cancer, an ovarian cancer, a prostate cancer, a liver cancer, a pancreatic cancer, a uterine cancer, a bladder cancer, a stomach cancer, a cervical cancer, a gallbladder cancer, an endometrial cancer, or a breast cancer.

32. The method of any one of claims 24-26 or the kit of any one of claims 29-31, wherein the patient is a human.

33. A method of making an antibody drug conjugate (ADC), the method comprising conjugating an antibody to a cytotoxic or cytostatic agent via a linker,
wherein the antibody comprises a heavy chain variable region and a light chain variable region, wherein the heavy chain variable region comprises a CDR-H1 comprising the sequence of SEQ ID NO:12, a CDR-H2 comprising the sequence of SEQ ID NO:13, and a CDR-H3 comprising the sequence of SEQ ID NO:14; and wherein the light chain variable region comprises a CDR-L1 comprising a sequence selected from the group consisting of SEQ ID NOs: 15-17, a CDR-L2 comprising the sequence of SEQ ID NO: 18, and a CDR-L3 comprising the sequence of SEQ ID NO: 19.

34. The method of claim 33, wherein the ADC further comprises a spacer between the linker and the antibody, and/or a spacer between the linker and the cytotoxic or cytostatic agent.

35. An ADC produced by the method of claim 33 or claim 34.


SEQUENCE LISTING (20 sequences)

SEQ ID NO: 1 - protein, 115 aa, synthetic construct
EVQLVESGGGLVQPGGSLRLSCAASGFTFSTNAMSWVRQAPGKGLEWVSRLRPKSDNYATYYADSVKGRFTISRDNSKNTLYLQMNSLRAEDTAVYYCAKGTGFWGQGTTVTVSS

SEQ ID NO: 2 - protein, 115 aa, synthetic construct
EVQLVESGGGLVQPGGSLRLSCAASGFTFSTNAMSWVRQAPGKGLEWVARLRPKSDNYATYYADSVKGRFTISRDNSKNTLYLQMNSLRAEDTAVYYCVTGTGFWGQGTTLTVSS

SEQ ID NO: 3 - protein, 115 aa, synthetic construct
EVQLVESGGGLVQPGGSLRLSCAASGFTFSTNAMSWVRQAPGKGLEWVARLRPKSDNYATYYADSVKGRFTISRDDSKNTLYLQMNSLRAEDTAVYYCVTGTGFWGQGTTLTVSS

SEQ ID NO: 4 - protein, 115 aa, synthetic construct
EVQLVESGGGLVQPGGSLRLSCAASGFTFSTNAMSWVRQAPGKGLEWVARLRPKSDNYATYYADSVKGRFTISRDDSTSTLYLQMNSLRAEDTAVYYCVTGTGFWGQGTTLTVSS

SEQ ID NO: 5 - protein, 113 aa, synthetic construct
DIVMTQSPDSLAVSLGERATINCKSSQSLLNSGNQKNYLTWYQQKPGQPPKLLIYWTSTRESGVPDRFSGSGSGTDFTLTISSLQAEDVAVYYCQNDYTSPYTFGQGTKLEIK

SEQ ID NO: 6 - protein, 113 aa, synthetic construct
DIVMTQSPDSLAVSLGERVTMNCKSSQSLLNSGNQKNYLTWYQQKPGQPPKLLIYWTSTRESGVPDRFSGSGSGTDFTLTISSVQAEDVAVYYCQNDYTSPYTFGQGTKLEIK

SEQ ID NO: 7 - protein, 113 aa, synthetic construct
DIVMTQSPDSLAVSLGERATINCKSSQSLLNSGNQKNYLTWYQQKPGQPPKLLFYWTSTRESGVPDRFSGSGSGTDFTLTISSLQAEDVAVYYCQNDYTSPYTFGQGTKLEIK

SEQ ID NO: 8 - protein, 113 aa, synthetic construct
DIVMTQSPDSLAVSLGERVTMNCKSSQSLLNSGNQKNYLTWYQQKPGQPPKLLFYWTSTRESGVPDRFSGSGSGTDFTLTISSVQAEDVAVYYCQNDYTSPYTFGQGTKLEIK

SEQ ID NO: 9 - protein, 113 aa, synthetic construct
DIVMTQSPDSLAVSLGERVTMNCKSSQSLLNSGNQKNYLTWYQQKPGQPPKLLFYWTSTRESGVPDRFSGSGSGTDFTLTISSVQAEDLAVYYCQNDYTSPYTFGQGTKLEIK

SEQ ID NO: 10 - protein, 113 aa, synthetic construct
DIVMTQSPDSLAVSLGERVTMNCKSSQSLLQSGNQKNYLTWYQQKPGQPPKLLFYWTSTRESGVPDRFSGSGSGTDFTLTISSVQAEDLAVYYCQNDYTSPYTFGQGTKLEIK

SEQ ID NO: 11 - protein, 113 aa, synthetic construct
DIVMTQSPDSLAVSLGERVTMNCKSSQSLLSSGNQKNYLTWYQQKPGQPPKLLFYWTSTRESGVPDRFSGSGSGTDFTLTISSVQAEDLAVYYCQNDYTSPYTFGQGTKLEIK

SEQ ID NO: 12 - protein, 10 aa, synthetic construct
GFTFSTNAMS

SEQ ID NO: 13 - protein, 12 aa, synthetic construct
RLRPKSDNYATY

SEQ ID NO: 14 - protein, 6 aa, synthetic construct
VTGTGF

SEQ ID NO: 15 - protein, 17 aa, synthetic construct
KSSQSLLNSGNQKNYLT

SEQ ID NO: 16 - protein, 17 aa, synthetic construct
KSSQSLLQSGNQKNYLT

SEQ ID NO: 17 - protein, 17 aa, synthetic construct
KSSQSLLSSGNQKNYLT

SEQ ID NO: 18 - protein, 7 aa, synthetic construct
WTSTRES

SEQ ID NO: 19 - protein, 9 aa, synthetic construct
QNDYTSPYT

SEQ ID NO: 20 - protein, 6 aa, synthetic construct
AKGTGF